(19) United States
(12) Patent Application Publication
(10) Pub. No.: US 2024/0389688 A1
(43) Pub. Date: Nov. 28, 2024

(54) PROCESS FOR REMOVING PROTEIN CONTENTS FROM RUBBER GLOVE AND RUBBER GLOVE PRODUCT RESULTING THEREFROM

(71) Applicant: Sri Trang Gloves (Thailand) Public Company Limited, Songkhla (TH)

(72) Inventors: Jarinya Jirojkul, Songkhla (TH); Sirithorn Kaewklum, Songkhla (TH); Parisa Faibunchan, Songkhla (TH)

(73) Assignee: Sri Trang Gloves (Thailand) Public Company Limited, Songkhla (TH)

(21) Appl. No.: 18/690,154

(22) PCT Filed: Oct. 14, 2021
(86) PCT No.: PCT/TH2021/000062

§ 371 Date: Mar. 7, 2024

(51) Int. Cl. A41D 19/00; B29C 71/00; C08C 1/04

(52) U.S. Cl. CPC A41D 19/0055; B29C 71/0009; C08C 1/04; B29C 2071/0027; B29C 2071/0045

(57) ABSTRACT

A process for removing protein contents from a rubber glove includes heating the rubber glove in water for 10-30 minutes; heating the rubber glove in a sodium carbonate solution for 50-70 minutes; and washing the rubber glove at least 2 times in washing water.

### FIELD OF THE INVENTION

The present disclosure relates to a process for removing protein contents from a rubber glove and the rubber glove product resulting from such process.

### BACKGROUND OF THE INVENTION

A conventional process for manufacturing rubber gloves from natural rubber or natural latex results in glove products having protein residues. When contacted with the user's skin, said residual proteins are the major cause of allergy and other adverse effects. These undesirable effects have led to the use of alternatives such as nitrile gloves which are made of synthetic rubber and have a lower risk of allergy. Still, latex gloves are preferred for their physical properties which provide superior comfort to the user. For users in the medical sector, latex gloves also afford a better protection against microorganisms.

Conventionally, a process for manufacturing said rubber gloves involves steps for removing residual protein contents from the glove products. Also conventionally, said protein removal is carried out by way of chlorination which involves the use chlorine, a toxic chemical species, and thus requires several working steps in order to handle the toxicity. Regardless, chlorination fails to reduce the protein contents down to a desirable level and the risk of allergy still persists.

The Japanese patent publication No. JPH07289564A disclosed a process aiming to reduce protein contents from gloves by washing finished glove products with water for multiple times at various water temperatures. While this publication claimed that the process was capable of reducing the protein contents down to a tenth of the protein contents found in then-conventional surgical gloves, such level of protein contents still amounted to a significant risk of allergy.

Further, the People's Republic of China's patent publication No. CN102059810A disclosed a production process for medical low-protein rubber gloves comprising steps of stirring the gloves in de-proteinization extraction solution formed by borax, ammonia water and water, before cooling to normal temperature and clear water washing. The process involved several working steps which took more than 24 hours in order to reduce the protein contents of the prototype glove to be at 53.9 percent of the initial protein contents.

Despite the foregoing previous attempts, there is still a need for a process that is simpler and more effective in the removal of protein contents from rubber gloves, including latex gloves.

### SUMMARY OF THE INVENTION

An object of the present invention is to provide a process that is capable of addressing the above need for a simpler and more effective removal of protein contents from rubber gloves, including latex gloves. Also, an object of the present invention is to provide a rubber glove product of desirable level of protein contents resulting from said process.

The present inventors have discovered that a process according to the present invention is surprisingly simple and effective for said objects. Also, the present inventors have found that the resulting rubber glove products or latex glove products contains desirable levels of proteins. Further, the present inventors have tested for the protein contents in the resulting glove products according to all the applicable industrial/medical standards, specifically: the standards for total proteins, for antigenic proteins, and for the four major allergens. All the tests, to be explained below, yielded remarkable results.

In the first aspect, an embodiment is a process for removing protein contents from a rubber glove. Said process comprises steps of: heating the rubber glove in water for 10-30 minutes; heating the rubber glove in a base solution for 50-70 minutes; and washing the rubber glove at least 2 times in washing water.

Preferably, the rubber glove is heated in the water at a temperature of 50-70° C.

Also preferably, the rubber glove is heated in the base solution at a temperature of 50-70° C.

Also preferably, the base solution is an alkaline or alkaline earth solution. More preferably, the base solution is a sodium carbonate solution. Even more preferably, the base solution is a solution of 0.1 to 0.3% sodium carbonate.

Also preferably, the washing water is at a temperature of 50-70° C.

Also preferably, the rubber glove is washed in the washing water at least 2 times, each of the times for 10-30 minutes.

Also preferably, said process further comprises a step of drying the rubber glove.

The above embodiments are preferably carried out after the rubber glove has been formed, and the form and structure of the rubber glove has been set and thus is essentially unaffected by the foregoing operating parameters. For this reason, the embodiments are preferably carried out after the rubber glove has been stripped.

Owing to its simplicity, the foregoing embodiments can be carried out within 2 hours or less and can be incorporated into any larger manufacturing process as a batch, continuous or semi-continuous process sub-component. In addition, the embodiments involve a base solution, which is most preferably a rather mild solution of 0.1 to 0.3% sodium carbonate, thereby solving the toxicity problem which had been vexing the state of the art's chlorination process.

The rubber glove resulting from the foregoing embodiments contains total proteins of less than 7 microgram/dm², or an assay concentration of total proteins of less than 2.2 microgram/milliliter, according to ASTM D5712-15 standard.

Also, the rubber glove resulting from the foregoing embodiments contains total proteins of less than 6 microgram/dm², or an assay concentration of total proteins of less than 2.0 microgram/milliliter, according to EN455-3 standard.

Further, the rubber glove resulting from the foregoing embodiments contains unquantifiable major allergens according to ASTM D7427-16 standard.

Even further, the rubber glove resulting from the foregoing embodiments contains antigenic proteins of less than 0.30 microgram/dm² according to ASTM D6499-18 standard.

Optionally, the rubber glove involved in the above embodiments is a natural rubber latex glove.

In the second aspect, an embodiment is a rubber glove product obtained from any of the above-described embodiments in accordance with the first aspect. Preferably, said rubber glove product contains non-detectable proteins. Also preferably, said rubber glove product is a natural rubber latex glove.

### BRIEF DESCRIPTION OF DRAWINGS

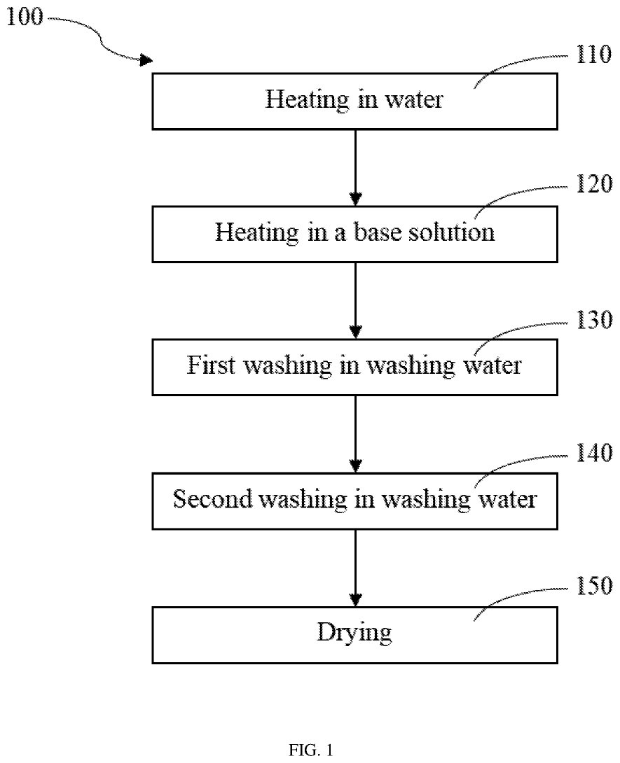

The principle of the present invention and its advantages will become apparent in the following description, taking into consideration the accompanying drawing in which: FIG. 1 shows a schematic flowchart of a process in accordance with a preferred embodiment.

### DETAILED DESCRIPTION OF EMBODIMENTS OF THE INVENTION

It is to be understood that the following detailed description will be directed to embodiments, provided as examples for illustrating the concept of the present invention only. The present invention is in fact not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section.

Unless defined otherwise, all technical and scientific terms used herein have the same meaning as commonly understood by one of ordinary skills in the art to which this invention belongs.

It must be noted that as used herein and in the appended claims, the singular forms “a”, “an”, and “the” include plural referents unless the context clearly dictates otherwise.

The term “about” when used before a numerical designation, e.g., dimensions, time, amount, and such other, including a range, indicates approximations which may vary by (+) or (−) 10%, 5% or 1%, or any sub-range or sub-value there between.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a device or method consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

In this application, “rubber glove” refers to a glove made of natural rubber or natural latex, and is to be distinguished from a glove made of synthetic latex or synthetic rubber, such as a nitrile glove or neoprene glove.

In this application, “base solution” refers to a liquid solution which exhibits a basic property, i.e. having a pH greater than 7, wherein the solute in an ambient condition may be in the solid, liquid or gas state and may be an organic or inorganic compound, and wherein the solvent may be water or any other appropriate chemical species.

In this application, “washing water” refers to any of tap water, distilled or otherwise purified water, with or without impurities or additives such as detergent or water softener/hardener, so long as such consists essentially of water and is considered appropriate for the washing carried out in an industrial facility, and where the rubber glove is of a medical grade, for the washing of medical-grade industrial product. Said appropriateness may be determined by a person of normal skills in the relevant art.

### The Preferred Embodiment

FIG. 1 shows a schematic flowchart of a process in accordance with a preferred embodiment. Here, a process 100 for removing protein contents from a rubber glove is preferably carried out after the rubber glove has been stripped in accordance with a known stripping process and also preferably after the rubber glove is weighed. The process 100 comprises subcomponent steps of heating the rubber glove in water 110, heating the rubber glove in a base solution 120, first washing of the rubber glove in washing water 130, second washing of the rubber glove in washing water 140, and drying the rubber glove 150.

The subcomponent steps of heating the rubber glove in water 110, heating the rubber glove in a base solution 120, first washing of the rubber glove in washing water 130, and second washing of the rubber glove in washing water 140, may take place in different or same vessel according to circumstantial design choices. The minimum requirement for a vessel for any of the foregoing steps is that such must be suitable for containing water, washing water and base solution at the relevant temperatures. Preferably, such vessel is capable to heat its content to the relevant temperatures. The vessel is also preferably suitable for exerting mechanical agitation, such as shaking and rotation, upon its content.

Examples of vessels suitable for the subcomponent steps of heating the rubber glove in water 110, heating the rubber glove in a base solution 120, first washing of the rubber glove in washing water 130, and second washing of the rubber glove in washing water 140, are inclusive of but not limited to, a tank or a drum equipped with a means for controlling temperature of its content. Said vessel is also preferably equipped with a means for shaking, turning or rotating its content, in order to promote the mixing during the immersion of the glove in a liquid.

Examples of equipment suitable for the subcomponent step of drying the rubber glove 150 are inclusive of but not limited to a drying hopper or oven of which the means for heating involves any of steam-heating coil, electricity, and heated air. Said drying equipment is preferable equipped with a means for shaking, turning or rotating its content, in order to promote the exposure of the glove's surface to the drying medium.

After glove has been stripped, the glove is preferably weighed so as to achieve a proportion of glove's weight that is within a range of 0.05-0.15 kg of glove per 1 liter of water, base solution or washing water used in the process, as the case may be.

Preferably, in heating the rubber glove in the water 110, the rubber glove is heated in the water at a temperature of 50-70° C., and also preferably for 10-30 minutes.

Also preferably, in heating the rubber glove in the base solution 120, the rubber glove is heated in the base solution at a temperature of 50-70° C., and also preferably for 50-70 minutes.

Also preferably, the base solution is an alkaline or alkaline earth solution. More preferably, the base solution is a sodium carbonate solution. Even more preferably, the base solution is a solution of 0.1 to 0.3% sodium carbonate.

Also preferably, in the first and second washings of the rubber glove in washing water 130, 140, the washing water is at a temperature of 50-70° C.; and also preferably, said steps 130, 140 are carried out for 10-30 minutes.

More preferably, a rotation is applied to at least one of the heating steps 110, 120 and washing steps 130, 140 at a speed of 10-20 revolutions per minute.

In some embodiments, the washing steps 130, 140 are carried out successively and with the same duration, temperature of washing water, and rotation speed. In such embodiments, it is preferred that at the first washing 130 and second washing 140 do not use the same washing water.

Also preferably, the drying step is carried out at a temperature of 80-130° C., and also preferably for 50-70 minutes.

### Example of a Preferred Embodiment

A process in accordance with a preferred embodiment was run according to the below particulars:

In this example, the rubber gloves were powder-free, polymer coated, and non-sterile natural rubber latex gloves. Said gloves had been stripped in accordance with a known stripping process, dried, and then weighed to 100 kg.

In this example, the step of heating the rubber gloves in water 110 was carried out by heating said gloves in 1,000 liters of filtered tap water at 65° C. for 20 minutes in a rotatable drum, during which a rotation at a speed of 15 revolutions per minute was also applied. Afterwards, the filtered tap water was drained from said rotatable drum.

Next, the step of heating the rubber gloves in a base solution 120 was carried out. First, 1,000 liters of filtered tap water was filled into the rotatable drum, still containing the gloves from the previous step, then the filtered tap water was heated up to the temperature 65° C. Next, a 19-21% by weight solution of sodium carbonate in filtered tap water of substantially same temperature, prepared from sodium carbonate powder, was added into the rotatable drum with appropriate mild stirring, resulting in a 0.1% by weight solution of sodium carbonate. Said temperature of the 0.1% by weight sodium carbonate solution was maintained for 60 minutes in the rotatable drum, during which a rotation at a speed of 15 revolutions per minute was also applied. The sodium carbonate powder used in this example contained, in dry weight basis, 99.2% or more of total alkalinity (as sodium carbonate), 0.70% or less of total chloride content (as sodium chloride), 0.003% or less of iron content, 0.03% or less of sulphate content, and 0.03% or less of water insoluble matters. Said sodium carbonate powder was commercially sourced from Tianjin Red Triangle International Trading Co., Ltd. Afterwards, the sodium carbonate solution was drained from said rotatable drum.

Next, the step of first washing of the rubber gloves in washing water 130 was carried out. One thousand liters of filtered tap water was filled into the rotatable drum, still containing the gloves from the previous step. The filtered tap water was then heated up to the temperature 65° C. Said temperature was maintained for 20 minutes, during which a rotation at a speed of 15 revolutions per minute was also applied. Afterwards, the filtered tap water was drained. Subsequently, the step of second washing of the rubber gloves in washing water 140 was carried out by repeating the foregoing procedures for another 20 minutes before draining the filtered tap water.

Next, the gloves were removed from the rotatable drum and loaded into a drying oven, wherein the drying of rubber gloves 150 was carried out at a temperature of 100° C. for 60 minutes. The dried gloves were then left to cool down to the room temperature before removed from the drying oven for inspection.

### Inspection of Glove Resulted from a Preferred Embodiment

The above example of preferred embodiment was run in 3 repetitions, and the resulting gloves were subsequently sampled and inspected under the following standards: ASTM D5712-15, EN455-3, ASTM D7427-16, and ASTM D6499-18. Said inspections under said standards were also run for “Comparative Samples,” which represented the state of the art: the Comparative Samples were gloves of the same type, having proteins removed by way of conventional chlorination. The inspection results are provided in the following Tables 1-4:

| | Assay | | |
|---|---|---|---|
| | Concentration | Total proteins | |
| Sample Description | (μg/mL) | (μg/g) | (μg/dm²) |
| Comparative Sample | 8 | 40 | 25 |
| Sample 1 from the Preferred | BD | <11 | <7 |
| Embodiment | | | |
| Sample 2 from the Preferred | BD | <11 | <7 |
| Embodiment | | | |
| Sample 3 from the Preferred | BD | <11 | <7 |
| Embodiment | | | |

Table 2 below shows the results of inspection according to EN455-3, a standard for total protein contents. The indication of “BD” represents “below detection limit”. In accordance with said standard, the detection limit is 2.0 ug/mL.

| | Assay | | |
|---|---|---|---|
| | Concentration | Total proteins | |
| Sample Description | (μg/mL) | (μg/g) | (μg/dm²) |
| Comparative Sample | 4 | 18 | 12 |
| Sample 1 from the Preferred | BD | <10 | <6 |
| Embodiment | | | |
| Sample 2 from the Preferred | BD | <10 | <6 |
| Embodiment | | | |
| Sample 3 from the Preferred | BD | <10 | <6 |
| Embodiment | | | |

Table 3 below shows the results of inspection according to ASTM D7427-16, a standard for major allergens: Hev b1, Hev b3, Hev b5, and Hev b6.02. The indication of “UQ” represents “unquantifiable”. In accordance with said standard, the “quantifiable” contents of Hev b1, Hev b3, Hev b5, and Hev b6.02 are, respectively: 0.05 ug/g or more; 0.05 ug/g or more; 0.025 ug/g or more; and 0.025 ug/g or more.

| | Hev | Hev | Hev | Hev | |
|---|---|---|---|---|---|
| | b1 | b3 | b5 | b6.02 | Total |
| Sample Description | (μg/g) | (μg/g) | (μg/g) | (μg/g) | (μg/g) |
| Comparative Sample | UQ | UQ | 0.042 | 0.285 | 0.327 |
| Sample 1 from the | UQ | UQ | UQ | UQ | UQ |
| Preferred Embodiment | | | | | |
| Sample 2 from the | UQ | UQ | UQ | UQ | UQ |
| Preferred Embodiment | | | | | |
| Sample 3 from the | UQ | UQ | UQ | UQ | UQ |
| Preferred Embodiment | | | | | |

Finally, Table 4 below shows the results of inspection according to ASTM D6499-18, a standard for antigenic proteins. The indication of “BD” represents “below detection limit”. In accordance with said standard, the detection limit is 0.03 ug/mL.

| | Assay | | |
|---|---|---|---|
| | Concentration | Total proteins | |
| Sample Description | (μg/mL) | (μg/g) | (μg/dm²) |
| Comparative Sample | 0.10 | 0.50 | 0.30 |
| Sample 1 from the Preferred | 0.07 | 0.40 | 0.20 |
| Embodiment | | | |
| Sample 2 from the Preferred | BD | <0.20 | <0.10 |
| Embodiment | | | |
| Sample 3 from the Preferred | BD | <0.20 | <0.10 |
| Embodiment | | | |

The above Tables 1-4 show that a process in accordance with an embodiment and rubber glove products resulting therefrom exhibited results which were distinctly and surprisingly improved over a chlorination process in accordance with the state of the art.

1. A process for removing protein contents from a rubber glove, comprising:
heating the rubber glove in water for 10-30 minutes;
heating the rubber glove in a sodium carbonate solution for 50-70 minutes; and
washing the rubber glove at least 2 times in washing water.

2. The process according to claim 1, wherein the rubber glove is heated in the water at a temperature of 50-70° C.

3. The process according to claim 1, wherein the rubber glove is heated in the sodium carbonate solution at a temperature of 50-70° C.

4. (canceled)

5. The process according to claim 1, wherein the sodium carbonate solution is a solution of 0.1 to 0.3% sodium carbonate.

6. The process according to claim 1, wherein the washing water is at a temperature of 50-70° C.

7. The process according to claim 1, wherein the rubber glove is washed in the washing water at least 2 times, each of the times for 10-30 minutes.

8. The process according to claim 1, further comprising a step of drying the rubber glove.

9. The process according to claim 1, said process being carried out after the rubber glove has been stripped.

10. A process for removing protein contents from a rubber glove, said process comprising steps of:
heating the rubber glove in water at a temperature of 50-70° C. for 10-30 minutes;
heating the rubber glove in a solution of 0.1 to 0.3% sodium carbonate at a temperature of 50-70° C. for 50-70 minutes; and
washing the rubber glove at least 2 times in washing water, each of the times at a temperature of 50-70° C. for 10-30 minutes.

11. The process according to claim 10, further comprising a step of drying the rubber glove.

12. The process according to claim 10, said process being carried out after the rubber glove has been stripped.

13. The process according to claim 1, wherein the rubber glove resulting from said process contains total proteins of less than 7 microgram/dm² according to ASTM D5712-15 standard.

14. The process according to claim 1, wherein the rubber glove resulting from said process contains an assay concentration of total proteins of less than 2.2 microgram/milliliter according to ASTM D5712-15 standard.

15. The process according to claim 1, wherein the rubber glove resulting from said process contains total proteins of less than 6 microgram/dm² according to EN455-3 standard.

16. The process according to claim 1, wherein the rubber glove resulting from said process contains an assay concentration of total proteins of less than 2.0 microgram/milliliter according to EN455-3 standard.

17. The process according to claim 1, wherein the rubber glove resulting from said process contains unquantifiable major allergens according to ASTM D7427-16 standard.

18. The process according to claim 1, wherein the rubber glove resulting from said process contains antigenic proteins of less than 0.30 microgram/dm² according to ASTM D6499-18 standard.

19. The process according to claim 1 wherein said rubber glove is a natural rubber latex glove.

20. A rubber glove product containing non-detectable proteins obtained from the process according to claim 1.

21. The rubber glove product according to claim 20 that is a natural rubber latex glove.
